(19) United States
(12) Patent Application Publication
(10) Pub. No.: US 2011/0268753 A1
(43) Pub. Date: Nov. 3, 2011

(54) PROCESS FOR REGULATING IMMUNE RESPONSE

(75) Inventors: Robert P. Kimberly, Birmingham, AL (US); Jeffrey C. Edberg, Birmingham, AL (US); Jianming Wu, Vertavia Hills, AL (US); Kailhong Su, Omaha, NE (US)

(73) Assignee: The UAB Research Foundation, Birmingham, AL (US)

(21) Appl. No.: 13/143,835

(22) PCT Filed: Jan. 8, 2010
(86) PCT No.: PCT/US10/20493

§ 371 Date: Jul. 8, 2011

Related U.S. Application Data
(60) Provisional application No. 61/143,288, filed on Jan. 8, 2009.

(51) Int. Cl. A61K 39/00; A61P 37/00; C12Q 1/02

(52) U.S. Cl. 424/184.1; 435/29

(57) ABSTRACT

The discovery of FcγRIIc expression on B-cells allows several new methods of prediction or regulation of immune responses. A process of altering an immune response in a subject is provided by altering the expression level or activity of FcγRIIc on a cell. The relative ratio of activating FcγRIIc to inhibitory FcγRIIb levels in an immune cell allows prediction of the presence or absence of immune disease or abnormality such as rheumatoid arthritis or systemic lupus erythematosus. Inventive processes are provided whereby the relative levels of activating to inhibitory receptor expression in a subject is compared to an established inventive classification system to predict an immune response to a therapeutic, the presence or absence of disease, or the magnitude, duration, or timing of an immune response in the subject.

### CROSS-REFERENCE TO RELATED APPLICATIONS

This application claims priority from U.S. Provisional Application No. 61/143,288 filed Jan. 8, 2009, the entire contents of which are incorporated herein by reference.

### GRANT REFERENCE

The research carried out in connection with this invention was supported in part by grants from the NIH/National Institute of Arthritis, Musculoskeletal and Skid Diseases (R01 AR42476 and R01 AR49084).

### FIELD OF THE INVENTION

The invention relates generally to a process of affecting an immune response or predicting an immune response in a subject. More specifically, the invention relates to regulating cellular responses to activation or inhibition of Fc receptors and in particular to the function of FcγRIIc protein on B-cell function and the associated effects on cellular and systemic immunity. The invention is directed to diagnosing a disease or abnormality or predicting a propensity for a disease or response to a therapeutic by prediction of the magnitude, extent, or time of an immune response in a subject.

### BACKGROUND OF THE INVENTION

Immune responses are regulated through a delicate balance of activating and inhibitory forces that regulate the degree of response to foreign material in a subject. Shifting this balance in either direction produces disease such as rheumatoid arthritis (RA), systemic lupus erythematosus (SLE), Kawasaki disease, B-cell malignancies, autoimmune cytopenias, Evans syndrome, antiphospholipid syndrome, immune diabetes, and other immunoregulated diseases. Chronic inflammation and tissue damage are common when the tight regulation of immune response is imbalanced. Dijstelbloem H M, et al. Trends Immunol, 2001; 22:510-516; Salmon, J E, and Pricop, L, Arthritis Rheum, 2001; 44:739-750; Ravetch, J V, and Bolland, S, Annu Rev Immunol, 2001; 19:275-90.

Humoral and cellular factions of the immune system are linked by three families of receptors for IgG. These Fcγ receptors (FcγR) bind the constant region of IgG and are triggered by the presence of IgG-containing immune complexes. Depending on the cell type, cellular maturation stage, and magnitude of the response, signaling through FcγR affects cellular responses such as phagocytosis, antibody-dependent cellular cytotoxicity, cellular maturation or apoptosis, and release of inflammatory mediators. (see Wijngaarden, S, et al, Arthritis Rheum, 2004; 50:3878-3887.)

FcγRs are integral membrane proteins that are present on numerous cell types in a cell type restricted display on cells including B-cells, dendritic cells (DC), monocytes, macrophages, neutrophils, platelets, and natural killer cells. Ravetech, 2001. The role of dendritic cells in conferring both immunity and tolerance suggests this cell type as a primary source for immunoregulation. Mellman, I, and Steinman, R M, Cell, 2001; 106:255-258. Additionally, monocytes/macrophages are considered primary targets for modulating chronic inflammation in R A, Wijngaarden, 2004; Mulherin, D. et al, Arthritis Rheum, 1996; 39:115-124; Burmester, G R, et al, Arthritis Rheum, 1997; 40:5-18; Tak P P, et al, Arthritis Rheum, 1997; 40:215-225, as IgG-containing immune complexes that activate FcγR are present in high amounts in serum and inflamed joints of RA patients. Winchester, R J, et al, Clin Exp Immunol, 1970; 6:689-706; Mannik, M, J Rheumatol Suppl, 1992; 32:46-49.

The FcγR family contains both activating and inhibitory receptor subtypes. Activating receptor types include types I (CD64), IIa (CD32a), and III (CD16). These are characterized by the presence of at least one intracellular tyrosine-based activation motif (ITAM). In contrast, the inhibitory IIb (CD32b) subtype is defined by an intracellular tyrosine-based inhibitory motif (ITIM) that downregulates cellular activation or controls the level of tyrosine phosphorylation of intracellular signaling mediators.

FcγRII is a 40 kDa glycoprotein that demonstrates low affinity for IgG. Three genes have been described that encode for FcγRII (a, b, and c) which, due to alternative splicing mechanisms, encode six transcripts including FcγRIIa1, a2, b1, b2, b3, and c. Brooks, D G, et al, J Exp Med, 1989; 170:1369-1385; Qiu W Q, et al, Science, 1990; 248:732-735. Structurally, the FcγRII proteins contain two IgG-like extracellular domains that are encoded by two exons, a transmembrane domain encoded by a separate exon, and a cytoplasmic domain encoded by three additional exons. Brooks, 1989; Qiu, 1990. Isoforms FcγRIIb1 and FcγRIIb2 possess independent cellular expression. For example, FcγRIIb1 is expressed predominantly on B-cells, whereas FcγRIIb2 is expressed on monocytes, macrophages, neutrophils, and eosinophils. Hulett, M D, and Hogarth, P M, Adv Immunol, 1994; 57:1-127.

FcγRII isoforms possess highly homologous extracellular domains, but differ in ligand specificity suggesting tight regulation of this sequence. Ravetech, J V, and Kinet J P, Annu Rev Immunol, 1991; 9:457-492; Capel, P J A, and van de Winkel, J G J, Immunol Today, 1993; 14:215-221; Hulett, M D, and Hogarth, M, Adv Immunol, 1994; 57:1-127. The cytoplasmic domains are more divergent in sequence leading to distinct signaling functions of the Ha and IIb isoforms. Ravetch, J V, Cell, 1994; 78:533-560; Lin, C T, et al, J Clin Immunol, 1994; 14:1-13. For example, activation by crosslinking of FcγRIIa isoform modulates intracellular Ca²⁺ concentration, phagocytosis, and internalization of immune complexes. Metes, D, et al, Blood, 1998; 91:2369-2380; Odin, JA, et al, Science, 1991; 254:1785-1788; Tuijnman, W, et al, Blood, 1992; 79:1651-1656; van den Herik-Oudijk, I E, et al, J Immunol, 1994; 152:574-585. In contrast, the level of cellular activation by antigen receptors of B-cells (BCR), T-cells (TCR), or another Fc receptor is downregulated by signaling through the FcγRIIb isoform. Muta, T, et al, Nature, 1994; 368:70-73 (Erratum in: Nature, 1994; 369:340); Daeron, M, et al, Immunity, 1995; 3:365-646.

Inhibitory FcγRIIb may serve either to prevent activation signals by FcγRIIa, or through its own signaling function in adjusting the magnitude of cellular response. Studies in rat suggest that homoaggregation of FcγRIIb does not produce effector functions. Daeron, M, et al, J Immunol, 1992; 149:1365-1373. However, heteroaggregation between FcγRIIb and FcγRIIa inhibits activation. Daeron, 1995. Thus, the ratio of activating to inhibitory FcγR alters the threshold by which IgG-containing immune complexes may activate FcγR-containing cells. Wijngaarden, 2004.

While the alternating characteristics of the activating and inhibitory FcγRII isoforms suggests that patients with lower functional levels of FcγRIIa or higher levels of FcγRIIb may be less susceptible to autoimmune diseases or that alteration of the balance would allow for therapeutic disease modulation, no direct correlation has been identifiable. Thus, there exists a need for processes of diagnosing and treating immune diseases.

### BRIEF DESCRIPTION OF THE DRAWINGS

FIG. 1 depicts the presence of FcγIIb protein and FcγIIc protein in EBV-infected human B-cells that do not express FcγIIa protein where the cells are expressing either open reading frame (ORF) FcγIIc or the pseudogene (STP) with the results from each detection of mRNA compared to that expressed in U937 cells;

FIG. 2 depicts the presence of FcγIIc protein expressed in EBV-infected B-cells as detected by immunopreciptating FcγIIc from cell lysates with an anti-FcγIIc antibody directed at the extracellular domain followed by immunoblotting with either an antibody to FcγIIa/c cytoplasmic tail or FcγIIb cytoplasmic tail to demonstrate the presence of both FcγIIb and FcγIIc protein expressed in the cells;

FIG. 3 depicts the expression of FcγIIc on the surface of B-cells where all surface proteins were biotinylated and all FcγIIc proteins were immunoprecipitated with a specific antibody followed by immunoblotting with an avidin-HRP molecule to visualize the presence of FcγIIc protein in the cell membrane;

FIG. 4 depicts immunofluorescence of FcγIIb and FcγIIc in resting or activated B-cells where the proteins are crosslinked by an anti-FcγIIb extracellular domain specific antibody;

FIG. 5 depicts the phosphorylation of FcγIIc protein by crosslinking it with BCR using IgG or F(ab′)₂ fragments thereof wherein the level of phosphorylation is increased to a maximum between one and three minutes and wherein corresponding phosphorylation of the intracellular signaling proteins Syk and BLNK show similar rates of activation and inactivation as FcγIIc protein indicating signaling specific to FcγIIc in B-cells;

FIG. 6 depicts the reversal of inhibition of Btk phosphorylation by expression of FcγRIIc B-cells wherein the presence of ORF FcγRIIc correlates with the increase in phosphorylation of Btk whereas in the absence of FcγRIIc expression (i.e. pseudogene (STP)) the level of Btk phosphorylation is inhibited;

FIG. 7 depicts the reversal of Ca²⁺ flux due to the presence of FcγRIIc;

FIG. 8 depicts the expression of forms of FcγRIIc on B-cells;

FIG. 9 depicts a schematic of the FCGRA, FCGR2B, and FCGR2C, as well as FCGR3A and FCGR3B with unique sites for pyrosequencing;

FIG. 10 depicts a ratio plot for the three FcγRII proteins in control and SLE patients.

### DETAILED DESCRIPTION OF THE INVENTION

The instant invention has utility in modulating immune response and disease by regulating the function or expression level of Fc receptors. The present invention has further utility as a treatment for a variety of diseases and conditions; and predicts an immune response or response to a potential therapeutic based on comparing a test subject immune ratio relative to a value classification system obtained by measuring responses or phenotype of multiple subjects with a range of manifestations extending between normal and a diseased or condition state.

The functional level of inhibitory FcγRIIb is indicative of the expression of autoimmunity. Particularly, FcγRIIb polymorphisms are linked with an SLE phenotype in the human population. Su, K, et al, J Immunol, 2004; 172:7186-7191. Decreases in functional expression of FcγRIIb may lead to autoimmunity. However, the presence of FcγRIIb and FcγRIIa is insufficient to account for cellular or other immune responses or resistance in some patients to treatment with therapeutics.

A FCGR2C gene is an unequal crossover between genes encoding FcγRIIa and FcγRIIb such that the extracellular and transmembrane domains are derived from the gene for FcγRIIb and most of the cytoplasmic tail is derived from the gene for FcγRIIa. Warmerdan P A M, et al, J Biol Chem, 1993; 268:7346-7349. Gene expression has been identified in monocytes, macrophages, and polymorphonuclear cells. Cassel, D L, et al, Mol Immunol, 1993; 30:451-460. However, the primary transcript previously identified contains a stop codon at amino acid 13 (nt202) in its extracellular domain. Thus, while gene expression has been found in some cell types, the gene is considered a pseudogene due to a lack of identified protein expression in B-cells.

It was previously demonstrated that increase in functional haplotypes of FcγRIIb associate with SLE. Su, 2004. For example, the FcγRIIb haplotype 2B.4 (−386C-120A) demonstrates significant association with p=0.0054 and an odds ratio of 1.65. Id. In contrast, no association is demonstrated between FcγRIIc alleles or haplotypes and autoimmune SLE (p=0.975). Therefore, it was unexpected that altering the expression level or function of FcγRIIc, as in the instant invention, leads to modulation of autoimmune diseases. Similarly, it was unexpected that calculating the ratio(s) of FcγRIIc to FcγRIIb protein number expression and/or activity on a cell, or to activating FcγRs can be used to predict the degree, magnitude, or extent of either an immune response, the response to a potential therapeutic in a subject, or the response to a potential therapeutic in vivo or in vitro. With a quantification of a total expression or activity of FcγRIIb and FcγRIIc, and optionally FcγRIIa coupled with the knowledge each plays, individualized immune response and therapy regimes are predicted relative to a classification system of receptor number and/or activity correlated with the: clinical status of multiple tested individuals having known clinical status as to normal or diseased, alone or in combination with multiple tested individual haplotype.

As used herein, the terms “disease” or “condition” refers to a chronic or acute abnormality illustratively including RA; other rheumatic disorders; SLE; Kawasaki disease; B-cell malignancies; T-cell malignancies; non-Hodgkins lymphoma, autoimmune cytopenias; idiopathic thrombocytopenic purpura; autoimmune hepatitis; periodontitis; Evans syndrome; antiphospholipid syndrome; immune diabetes; asthma, dermatomyositis; ANCA-associated vasculitides; organ transplant; inflammation; tissue damage; antibody based immunotherapy; renal inflammatory diseases; sepsis; viral infection illustratively including Ebola infection; bacterial infection; chronic urticaria; other immunoregulated diseases; immunization; allergic disease or reaction; or combinations thereof.

The term “immune response” refers to a kinetic or magnitude variation of one or more elements of a subject immune system. Non-limiting examples of immune responses include B-cell responses, calcium mobilization, calcium influx, or other changes in intracellular calcium concentrations in any cellular compartment illustratively including the cytoplasm; nitric oxide production or release; phagocytosis; immunoglobulin uptake; production of immunoglobulin; alteration of protein phosphorylation; conversion of immune complexes; alteration of serum immunoglobulin levels; modulating the activity of spleen tyrosine kinase (Syk), B-cell linker (BLNK), Burton's tyrosine kinase (Btk), Kit, Lck, Zap-70, Src, Stat1, SHP-2, phosphatidyl inositol 3-kinase (PI3K), phosphoinositol 5-phosphatase, other kinases or phosphatases known in the art, phospholipase D, phospholipase C, sphingosine kinase; secretion of IL-1β, IL-6, IL-10, IL-2, IL-4, IFN-γ, Bcl10, TCR, TLR, or other cytokines, chemokines, or signaling molecules; interferon signaling; alteration of expression of interferon response gene(s) (IRG); antibody production illustratively IgE or IgG production; alteration of the expression of any gene that encodes for a protein, as well as the functional activity of any protein listed in Table 1; alteration of expression or activity of My4+/LeuM3− molecule; protection from challenge after exposure to infectious organism; alteration in nitrite levels; B-cell responses in various immune compartments; lymphoma cell responses; natural killer cell responses; monocyte responses; macrophage responses; platelet responses; dendritic cell responses; any immune cell response; Th1 and Th2 cytokine responses in various immune compartments; immune cell maturation; activation or inhibition of an intracellular signaling pathway such as the NF-kappa B signaling pathway; apoptosis; alteration in allotype or isotype antibody levels; in vitro recognition of antigen; survival; other response known in the art; or combinations thereof.

TABLE 1
(adopted from Dhodapkar, KM, et al,
J Exp Med, 2007; 204: 1359-1369):
Illustrative Cytokines and Chemokines

| | |
|---|---|
| IL-1α | IL-13 |
| IL-1β | IL-15 |
| IL-2 | IFN-γ |
| IL-3 | TNF-α |
| IL-5 | Eotaxin |
| IL-6 | MCP1 |
| IL-7 | Rantes |
| IL-8 | MIP1a |
| IL-10 | IP10 |
| IL-12p40 | IFN-α |
| IL-12p70 | |

As used herein, a “subject” refers to a cell or organism expressing both FcγRIIb and FcγRIIc receptor proteins on a given cell. Organisms illustratively include: humans; non-human primates illustratively including monkey, chimpanzee, and others; horses; goats; cows; sheep; pigs; dogs; cats; guinea pigs; hamsters; rabbits; mice; rats; other rodents; or combinations thereof. A subject is illustratively a patient suffering a disease or condition. A subject that is a cell is preferably an immune cell. Optionally a cell is a leukocyte. B-cells are optionally subjects has used herein.

As used herein, the term “regulating” or “regulation” refers to altering expression of a gene by increasing transcription rate or level; decreasing transcription rate or level; altering an interaction between regulatory sequences upstream, downstream, or within a gene and effector proteins or nucleic acid molecules; increasing translation rate or level; decreasing translation rate or level; altering transcript splicing, the rate of transcript splicing, or the fidelity of transcript splicing; increasing, decreasing, or otherwise altering the interaction between DNA, RNA, or protein and a biological sequence of DNA, RNA, or protein; increasing or decreasing the affinity of a protein and a protein, DNA, or RNA sequence, or a ligand; increasing, decreasing, or otherwise altering the association of a protein and a ligand; increasing, decreasing, or otherwise altering the transmission functional activity of a protein, DNA, or RNA sequence whether the functional activity be stimulatory or inhibitory; increasing, decreasing, or otherwise altering the physical, chemical or other association or aggregation of protein illustratively including antigen receptors on B-cells (BCR), antigen receptors on T-cells (TCR), or any Fc receptor such as FcγRI, FcγRII, or FcγRIII; increasing, decreasing, or otherwise altering the physical, chemical or other association or aggregation of GATA4, YY1, Elf-1, other Ets family transcription factors, STAT-1 and other STAT family transcription factors, and other transcription factors known in the art with protein, DNA, or RNA; altering B-cell responses in various immune compartments; altering lymphoma cell responses; altering natural killer cell responses; altering monocyte or macrophage responses; altering platelet responses; altering dendritic cell responses; altering any immune cell response; altering any immune response; or combinations thereof. It is appreciated that other forms of biological regulation are known in the art and are recognized by and incorporated into the meaning of regulating as used herein.

As used herein, the term “expression” or “expressing” refers to gene transcription, translation, protein production, or protein display intracellularly, integral to a membrane, or associated with a membrane.

As used herein, the term “ligand” refers to any molecular entity capable of interacting with one or more Fc receptors; an immunoglobulin, an immunoglobulin bound to a molecule; C-reactive protein; Fibrinogen-like protein 2; and the like.

As used herein, the term “biological sample” refers to a substance containing at least one subject cell expressing both FcγRIIb and FcγRIIc thereon and illustratively includes whole blood, plasma, serum, extracellular fluid, cytosolic fluid, tissue, solubilized cellular membrane samples, cultured cells, cell culture media, and physiological buffered forms thereof.

As used herein the term “therapeutic” refers any molecule or therapy that affects or is effected by immune cells or immune mediators. Illustratively, a therapeutic is: radiation exposure; an antibody; an immunogen; an antigen; cytokines; interleukins; ES-62; and any chemotherapeutic listed in Strome, SE, et al, The Oncologist, 2007; 12:1084-1095, the entire contents of which are incorporated herein by reference. A therapeutic is preferably an antibody or antibody fragment. An antibody is optionally polyclonal or monoclonal, and antibody fragment, or a fusion protein. An antibody is optionally: antibody 2B6; antibody CC49, anti-FcoRI Fab; IgA complexe(s); antibody conjugates; rituximab; trastuzumab; cetuximab; Alemtuzumab; Omalizumab; and Abatacept. It is appreciated that an antibody is optionally modified to be tolerated by a subject. Optionally, an antibody is humanized by processes known in the art.

As used herein the term “cell” refers to a B-cell, T-cell, monocyte, macrophage, natural killer cell, dendritic cell, platelet, erythrocyte, mast cell, megakaryocyte, neutrophil, or an osteoclast and other eukaryotic cell that in at least some subjects express both FcγRIIb and FcγRIIc.

Immune responses are regulated by a delicate balance of Fcγ receptor activation that determines the outcome of immune response and immuno-directed therapies. Ravetech, 2001. While a gene encoding FcγRIIc is known and is expressed to mRNA in several cell types, the gene is believed in the art to be a pseudogene, particularly in B-cells (Su, et al., Genes & Immunity, 2002. 3:551-556). A thymine at nucleotide 202 results in a stop codon at amino acid 13 resulting in no translation of FcγRIIc into protein. The only report of FcγRIIc expression was in natural killer cells from a small percentage of donors. Metes, 1998. No study has reported expression of FcγRIIc protein on other cell types indicating that on most immune cell types FcγRIIc is a pseudogene and is not expressed on the cell surface as a functional receptor. Surprisingly, genotypic analyses of B-cell populations in humans identified that this site is polymorphic, with some individuals expressing a cytosine at position 202 encoding a glutamine at amino acid position 13 that results in a full length FcγRIIc protein expressed on B-cells. Thus, an individual polymorphic at codon 202 for cytosine has an open reading frame that yields a full length FcγRIIc protein on B-cells that modulates immune response on B-cells. As FcγRIIb expressed on B-cells has an extracellular domain with homology to FcγRIIb and cytoplasmic domain homology to FcγRIIa (ITAM), FcγRIIc when expressed on a B-cell has implications on the overall immune response of a B-cell to IgG as activated or inhibited. As such, by genotyping a B-cell for the presence of FcγRIIc a response of subject B-cells to an immune challenge can be predicted, thus, defining subjects that vary in FcγRIIc expression 202 T/C who can have opposite results of activation versus inhibition in response to the same challenge. With B-cells being a primary actor in therapeutic lymphoma targeting, B-cell genotyping for FcγRIIc is optionally highly beneficial to such targeting therapeutics.

In a preferred embodiment, the expression level of a gene encoding FcγRIIc is altered by increasing the FCG2C gene expression. Illustratively, immune cells are exposed to increased levels of GATA4 or YY1 transcription factors. Increases of either GATA4 or YY1 upregulates FcγRII promoter activity increasing gene expression. Su, 2004. Alternatively, DNA or mRNA encoding full length FcγRIIc is delivered to immune cells such as B-cells. Delivery systems are illustratively viral, immunoliposomes, ionic lipid coating; a coating of carbohydrate around a nucleic acid, bile acids, or other delivery systems known in the art.

Illustratively adenovirus (Ad) is used as a delivery vector. Viral selection is determined by considerations of viral vector tropism, sites of vector expression within a host cell, ease of vector gene manipulation, required duration of expression, pathogenicity and the like. The Ad affords many advantages as a vector as evidenced by its popularity. Ad replicates episomally within a host cell and as such the host cell genome is unaltered resulting in no transgene expression in host cell daughters. The adeno-associated virus (AAV) is a smaller virus than Ad, which is capable of integrating into a host cells chromosomes and thereby affords the option of long-term expression. Gene therapy has shown clinical success in several instances including treatment of ocular disease with AAV vectors. Bainbridge, JWH, et al., N Engl J Med. 2008; 358(21):2231-9; Maguire, AM, et al., N Engl J. Med., 2008; 358(21):2240-8. Extension of these techniques to transform B-cells are now within the purview of one of skill in the art.

Various promoter sequences are optionally incorporated into an expression vector encoding FcγRIIc. Promoter sequences are preferably selected based on cell, tissue, organ, or organism specific expression. Expression specific promoters are known in the art and are operable herein.

Any suitable process for increasing gene expression is operable herein. Illustratively, expression of the FCGRIIC gene is increased by exposure of cells to phorbal ester (PMA) or interferon-gamma (IFN-γ). Other processes of increasing protein expression are similarly suitable illustratively including decreasing protein degradation such as by exposure of cells to cyclohexamide (CX).

In a preferred embodiment the expression of FcγRIIc protein is decreased. A decrease in expression is illustratively achieved by lowering transcription of the FCGRIIC gene, decreasing translation of FcγRIIc mRNA to protein, increasing the degradation rate of FcγRIIc mRNA, increasing the degradation rate of FcγRIIc protein, or other processes known in the art. Preferably, translation of the FCGRIIC gene to mRNA is decreased. Illustratively, exposure of cells to actinomycin D (ActD) decreases expression of FCGRIIC.

Preferably, a direct process of selectively decreasing the levels of mRNA or the translation of mRNA to protein in a cell is employed. In a preferred embodiment, FcγRIIc protein expression is reduced. RNAi technology is optionally used to recognize and degrade mRNA from FcγRIIc, FcγRIIb, or FcγRIIa. B-cell or other immune cell specific knock down of FcγR proteins is illustratively achieved by exposing cells to an RNAi vector such as the BLOCK-iT vector (Invitrogen, Corp., Carlsbad, Calif.). It is appreciated that other vectors known in the art are similarly operable. An illustrative example of a B-cell specific promoter is the B29 B-cell specific minimal promoter as described by Omori, SA, and Wall, R, PNAS USA, 1993; 90:11723-11727, the contents of which are incorporated herein by reference. Techniques for producing, delivering, and employing a vector capable of downregulating the expression of protein in a tissue specific manner are described by Rao, M K, and Wilkinson, M F, Nature Protocols, 2006; 1:1494-1501, the contents of which are incorporated herein by reference.

Optionally expression of FcγRIIc is decreased by delivery of siRNA specific for FcγRIIc in B-cells. A B-cell specific antibody is CD19. Illustratively, a protamine coding sequence linked to the C-terminus of the CD19 heavy chain is produced similar to the methods of Song, E., et al., Nat. Biotechnol., 2005; 23:709-717, the contents of which are incorporated herein by reference. Small (<30 nucleotides) double stranded RNA is specifically delivered to B-cells by binding to the CD19-protaimine complex and administered directly such as in cell culture, in a targeted fashion such as in direct injection, or systematically such as intravenously. The CD19 directs the siRNA to B-cells whereby mRNA encoding FcγRIIc is degraded reducing FcγRIIc protein expression. Similar methods are illustratively employed to specifically target specific B-cell populations or other specific cell types using antibodies, aptamer, or other agents specific for a cell-type specific surface protein or component.

In a preferred embodiment the functional level of FcγRIIc protein is altered. Function is illustratively upregulated or downregulated. In an illustrative example, FcγRIIc function is downregulated. Down regulation is illustratively achieved by exposing a cell expressing FcγRIIc to a ligand that prevents signaling via FcγRIIc. An illustrative ligand is an immunoglobulin that targets FcγRIIc. Interaction of FcγRIIc with an immunoglobulin illustratively prevents signaling via the FcγRIIc ITAM motif, homoaggregation of FcγRIIc, heteroaggregation of FcγRIIc with FcγRIIb, FcγRIIa, FcγRI isoforms, FcγRIII isoforms; TCR-ζ, FcεRI gamma-chains, or other integral or membrane bound receptor or molecule. Preferably, an inhibitory ligand prevents heteroaggregation of FcγRIIc with TCR-ζ or FcεRI gamma-chains. An immunoglobulin operable herein is illustratively an Fc domain that is optimized for binding to FcγRIIc. Processes of Fc optimization are illustrated in Stavenhagen, J B, et al, Cancer Res, 2007: 67:8882-8890.

Optionally an immune response is predicted by quantifying the amount of FcγRIIc and comparing that quantity to the expression number of FcγRIIb or FcγRIIa isoforms. Optionally, comparison is between isoforms present on the same cell. Optionally, FcγRIIb levels are compared to FcγRIIc levels and an immune response is predicted from the result of the comparing.

The immune response of a B-cell is complicated by the ITIM properties of FcγRIIb expressed in concert with FcγRIIc on a subject B-cell. According to the present invention five novel haplotypes of FcγRIIb are provided that vary in ITIM alone or when combined with expression of FcγRIIc on a subject B-cell which affords for the first time a predictive model of B-cell immune response and the use of B-cells in therapeutic targeting of lymphoma and other diseases involving B-cell operation. Haplotypes include −386/−120 G/T, haplotype 2B.1; C/T, haplotype 2B.2; G/A, haplotype 2B.3; C/A, haplotype 2B.4. These haplotypes are described in Su, Kaihong, et al., J. Immunol., 2004; 172:7186-7191 and functionally characterized in J. Immunol, 2004; 172:7192-7199 both of which are fully incorporated herein by reference. Previously, only two FcγRIIb haplotypes were known and provided an incomplete model of cellular ITIM response in B-cell as well as other FcγRIIb expressing cells. With the present invention elucidating the role of a heretofore unknown B-cell Fcγ receptor FcγRIIc and a set of novel FcγRIIb haplotypes, the diverse range of B-cell immune response is apparent.

A still further aspect of the present invention that provides a tertiary level of variation in B-cell response after receptor expression (primary) and haplotype (secondary) is copy number variation. Copy number variation is exploited therapeutically by quantifying FcγRIIb number on a cell and then exposing the cell to a quantity of anti-FcγRIIb antibody (Ab) to reduce the number of FcγRIIb receptors still operative to bind IgG. Preferably the anti-FcγRIIb Ab exposed to the cell is specific for the FcγRIIb haplotype. Accordingly, an inventive anti-FcγRIIb Ab is provided raised against each of the five novel FcγRIIb haplotypes. The production of a purified monoclonal antibody to the extracellular domain of a cellular receptor is well known to the art.

Any suitable process for quantifying FcγRIIb or FcγRIIc protein on a cell is operable herein. Processes of quantifying FcγR illustratively include, specific recognition by discriminating ligands, specific monoclonal antibodies conjugated to a label, an antibody sandwich technique, immunoprecipitation, western blotting, HPLC, mass spectroscopy, radioimmunoassay (RIA), combinations thereof, or other processes known in the art. Preferably, labeled antibodies are used to detect and quantify specific FcγR proteins on a cell surface. A fluorescent label on the antibody provides for detection in a fluorometric assay system. Preferably, a flow cytometer is used to detect fluorescently labeled antibodies on a cell. Flow cytometry offers numerous advantages including providing selectivity of both target protein and cell type. Illustratively, whole blood is used as a biological sample. Multiple antibodies with differing specificity are contacted with the biological sample. Flow cytometry is employed to select for specific cell types based on both light scattering and recognition by antibodies specifically directed to the target cell type. Moreover, cells of differing maturation or immunocharacteristics are isolated to restrict FcγR quantitation to a desired cell type. Illustratively, a FACScan flow cytometer (BD Biosciences, San Diego, Calif.) is used. It is appreciated that other instruments from other manufacturers are similarly operable.

In a preferred embodiment a radio-immuno assay (RIA) is used to quantify FcγRII protein on a cell. Numerous techniques for RIA are known in the art. Antibodies specific for particular FcγRII proteins are optionally conjugated to or constructed with a radioisotope such as ¹²⁵I, ¹³¹I, ³²P, ³H, ¹⁴C, or combinations thereof. It is appreciated that other radioactive labels are similarly operable. Processes for RIA are illustratively those of Williams, T E, et al, Biophys J, 2000; 79:1858-1866, which is incorporated herein by reference.

Antibodies specific for particular FcγR proteins illustratively include antibody ZZ18 (Santa Cruz Biotechnology, Santa Cruz, Calif.) that specifically recognizes FcγRIIa; FcγRI is specifically recognized by antibody 32.2 (Medarex, Annandale, N.J.), FcγRIII is specifically recognized by antibody 3G8 (Medarex, Annandale, N.J.), TCR-ζ is recognized by antibody ab11281 (Abcam, Inc., Cambridge, Mass.), FcεRI gamma-chains is recognized by antibody 9E1 (Abcam, Inc., Cambridge, Mass.), and isotype matched controls are available from Immunotech (Marseilles, France). Antibody 4F5 is used to detect the presence of FcγRIIb. It is appreciated that numerous other antibodies, labeled or otherwise, are similarly operable to specifically or non-specifically recognize individual receptors for immunoglobulins. Preferably, antibodies listed recognize human receptor sequences. It is appreciated in the art that other antibodies are similarly operable for the other species encompassed by the term subject as used herein. Further, it is appreciated that the antibodies listed have reactivity toward receptors from numerous species. Antibodies for other species are illustratively available from Santa Cruz Biotechnologies, Santa Cruz, Calif.

Antibodies are preferably labeled. Labels operative herein illustratively include active esters (which include succinimidyl esters (SE), sulfosuccinimidyl esters (SSE), tetrafluorophenyl esters (TFP) and sulfodichlorophenol esters (SDP)), isothiocyanates (ITC), and sulfonyl chlorides (SC) (see Amine Reactive Probes, published by Molecular Probes, Eugene, Oreg.). Specific examples of operative labels are illustratively fluoroisothiocyanate (FITC), phycoerythrin (PE), Cy5, Cy3, Texas Red, Oregon Green, any dye listed in the table on page 8 of Amine Reactive Probes, published by Molecular Probes, Eugene, Oreg., which is incorporated herein by reference, any other label known in the art, or combinations thereof.

In a preferred embodiment cellular protein is quantified by immunoprecipitation and/or western blotting. Techniques for these detection processes are standard and known in the art. Illustrative techniques are available in Short Protocols in Molecular Biology, 5th Edition, John Wiley & Sons, Inc., Hoboken, N.J. Reagents for immunoprecipitation illustratively include antibodies directed to FcγRIIa, FcγRIIb, FcγRIIc, TCR-ζ or FcεRI. Specific antibodies directed to these molecules are illustrated herein. Antibodies operable herein are illustratively labeled. Suitable labels illustratively include horseradish peroxidase (HRP), alkaline phosphatase, fluorophores, combinations thereof, and other detection labels known in the art.

Other processes known in the art for detecting or quantifying protein in a biological sample are similarly operable herein. Illustratively, an enzyme linked immunoadsorbent assay (ELISA) is operable to both detect and quantify FcγRII proteins in a biological sample. Suitable reagents are available from companies such as Santa Cruz Biotechnology (Santa Cruz, Calif.), Invitrogen, Corp. (Carlsbad, Calif.), other companies stated herein, and numerous other sources known in the art. Preferably, and ELISA is a sandwich ELISA. It is appreciated that other ELISA protocols are similarly operable. Protocols for numerous ELISA formats are known in the art and are commonly available with assay kits or in Short Protocols in Molecular Biology, 5th Edition, John Wiley & Sons, Inc., Hoboken, N.J.

Additionally, FcγR protein quantity is illustratively achieved by correlation with FcγR specific mRNA levels. Numerous processes for quantifying mRNA levels are known in the art and are operable herein illustratively including RT-PCR, real-time PCR, probe hybridization, gel-electrophoresis, transcription specific assay, ribonuclease protection, combined ribonuclease protection with a scintillation proximity assay as described by Kenrick, MK, et al, Nucleic Acids Res, 1995; 25: 2947-2948, incorporated herein by reference, other processes known in the art, or combinations thereof.

In a preferred embodiment the level of FcγRIIc protein expression is compared the level of FcγRIIb protein on the same cell. FcγRIIc is an unequal crossover product between FcγRIIa and FcγRIIb. The extracellular and transmembrane domains of FcγRIIc are identical to FcγRIIb when they are expressed as part of the full length FcγRIIc isoform as in the instant invention and not the pseudogene. See e.g. Stuart, S G, et al, J Exp Med, 1987; 166:1668-1684; Stuart, S G, et al, EMBO J, 1989; 8:3657-3666; Warmerdam, P A M, et al, J Exp Med, 1990; 172:19-25, each of which are incorporated herein by reference. As such, full length FcγRIIc has similar immunoglobulin binding properties as FcγRIIb in B-cells.

The expression and functional activity of FcγRIIb on immune cells is decreased in patients with active SLE. Su, K, et al, J Immunol, 2007; 178:3272-80. However, in patients in remission from SLE the levels are not significantly different from normals. Id. The magnitude of the difference is 30% less in memory B-cells, and 15% less in plasma B-cells. Id. Thus, altering the functional activity of FcγRIIc sufficiently to produce equivalents to 5-95% the functional effect of reductions in FcγRIIb expression modulates an immune response and response to a potential therapeutic. In a preferred embodiment FcγRIIc levels are decreased between 1% and 99%. More preferably, FcγRIIc levels are decreased between 5% and 50%. Most preferably, FcγRIIc levels are decreased between 15% and 30%.

The type or magnitude of an immune response or response to a therapeutic is optionally predicted by determining optionally by quantifying the level of FcγRIIc on a cell, determining optionally by quantifying the level of FcγRIIb on a cell, calculating the ratio of FcγRIIb/FcγRIIc levels on the cell, and predicting the type or magnitude of an immune response or response to a therapeutic from the result of calculating. Optionally, predicting the type or magnitude of an immune response or response to a therapeutic is achieved by comparison of the immune ratio of FcγRIIb/FcγRIIc levels to a value classification system obtained by analyses of subjects that have a known immune response or response to a therapeutic. Similarity or dissimilarity optionally reveals the expected immune response or response to a therapeutic in the subject. Similarity is optionally within +/−100%, 75%, 50%, 25%, 10%, 5%, or 1% of a known ratio in the value classification system. Similarity is optionally within three standard deviations, two standard deviations, or one standard deviation of a known ratio in the value classification system. Optionally, gene copy number is used to quantify the level of FcγRIIc or inhibitory FcRs on a cell. A cell is illustratively a plurality of cells of the same or different types or lineages. In a preferred embodiment a cell is isolated from a biological sample. The biological sample is preferably obtained from a subject. Preferably, a biological sample is a sample of whole blood. Optionally, a cell is a B-cell. Optionally, the FcγRIIc/FcγRIIb ratio is between 0.01 and 100. Optionally, the FcγRIIc/FcγRIIb ratio is between 0.3 and 3. Optionally, the FcγRIIc/FcγRIIb ratio is between 0.5 and 2. It is appreciated that the term “level” refers to the copy number of each respective FcR, the copy number of genes encoding each FcR, the expression level of FcR mRNA or protein, or the concentration of FcR mRNA or protein. It is further appreciated that the term “level” optionally relates to an association to a predefined normal. It is further appreciated that the term “level” refers to a relative level between targets of interest.

In a preferred embodiment diagnosing a disease or abnormality, identifying propensity for a disease or abnormality, or both is illustratively achieved by obtaining a biological sample from a subject, isolating a cell or cell type from the subject, determining illustratively by quantifying the level of FcγRIIc on the cell, determining illustratively by determining optionally by quantifying the level of inhibitory FcRs, and calculating the ratio of FcγRIIc level to inhibitory FcRs level on the cell type from the subject. A cell is illustratively a plurality of cells of the same or different types or lineages. In a preferred embodiment a cell is isolated from a biological sample. The biological sample is preferably obtained from a subject. Optionally, a biological sample is a sample of whole blood. Optionally, a cell is a B-cell. The ratio calculated from the subject is compared to the ratio value classification system determined from a plurality of subjects that do or do not present the disease of interest. Diagnosing a disease or abnormality, identifying propensity for a disease or abnormality, or both is made from comparing the ratio from a subject to a value classification system obtained from a plurality of subjects with or without the disease or abnormality. Similarity or dissimilarity optionally reveals the expected absence or presence of the disease or abnormality of propensity for a disease or abnormality in the subject. Similarity is optionally within +/−100%, 75%, 50%, 25%, 10%, 5%, or 1% of a known ratio in the value classification system. Similarity is optionally within three standard deviations, two standard deviations, or one standard deviation of a known ratio in the value classification system. The comparing is optionally calculating the difference illustratively by subtraction, multiplication, addition, division, or other mathematical parameter such as a derivative determinable from numerous ratios calculated from quantifications taken over a period of time.

Activating FcR are illustratively FcγRI, FcγRIIa, FcγRIII, or other activating Fc receptors known in the art. Inhibitory FcR are illustratively FcγRIIb and other inhibitory Fc receptors known in the art.

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

Various aspects of the present invention are illustrated by the following non-limiting examples. The examples are for illustrative purposes and are not a limitation on any practice of the present invention. It will be understood that variations and modifications can be made without departing from the spirit and scope of the invention. While the examples are generally directed to mammalian tissue, a person having ordinary skill in the art recognizes that similar techniques and other techniques known in the art readily translate the examples to many organisms including humans. Reagents illustrated herein are commonly cross reactive between mammalian species or alternative reagents with similar properties are commercially available, and a person of ordinary skill in the art readily understands where such reagents may be obtained.

### Example 1

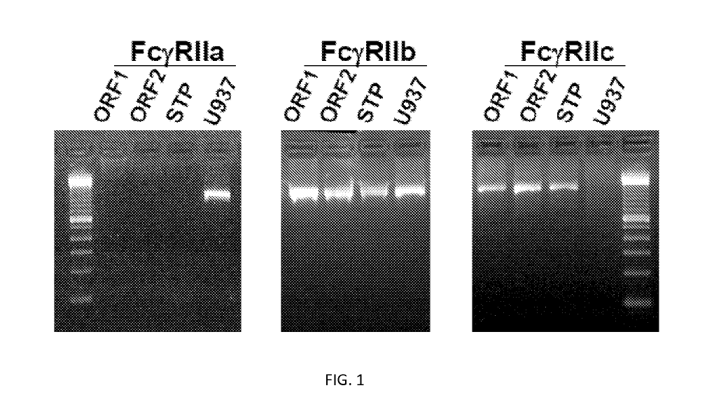

Detection of expressed FCGR gene products in multiple cell types. Multiple processes are optionally performed for the detection of gene products on various cell types. Briefly, as described by Su, et al. J Immunol, 2007; 178:3272-80, incorporated herein by reference, leukocyte subsets are enriched by cell specific microbeads and subsequently purified by FACS sorting after staining with cell surface specific markers. B-cells are enriched by CD19 microbeads (Miltenyi Biotec, Auburn, Calif.). NK cells are enriched by CD3-depletion and CD56 positive selection. Plasmacytoid dendritic cells (PDCs) are first enriched by BDCA4 microbeads using the Plasmacytoid Dendritic Cell Isolation Kit (Miltenyi Biotec, Auburn, Calif.) and then subjected to cell sorting for BDCA2-APC positive cells. Enrichment and isolation of other cell types are performed in protocols described by Miltenyi Biotec, Auburn, Calif., which are incorporated herein by reference. Analyses of the isolated cell populations by flow cytometry demonstrate sorted cell purity over 96%. Total RNA is prepared from the same number of sorted cells using Trizol Reagents (Invitrogen, Corp., Carlsbad, Calif.). The RT-PCR for transcripts from FCGR2B, FCGR2A, FCGR2C, and GAPDH (control) genes is performed using SuperScript III One-Step RT-PCR Kit (Invitrogen Corp., Carlsbad, Calif.) following the manufacturer's protocol. The FcγRIIb-specific sense primer is: 5′-TGTCCAAGCTCCCAACTCTTCACC-3′ (SEQ. ID. No. 1); the antisense primer is: 5′-GTGTTCTCAGCCCCAACTTTG-3′ (SEQ. ID. No. 2). The FcγRIIa-specific sense primer is: 5′-CACTGTCCAAGTGCCCAGCAT-3′ (SEQ. ID. No. 3); the antisense primer is: 5′-TTTATCATCGTCAGTAGGTGCCC-3′ (SEQ. ID. No. 4). The FcγRIIc-specific sense primer is: 5′-CGGAATTCTGATGGGAATCCTGTCAT-3′(SEQ. ID. No. 5). Metes, 1998. The FcγRIIc-specific antisense primer is: 5′-GCTCTAGATGACCACATGGCATAACG-3′ (SEQ. ID. No. 6). Id. RT-PCR reaction conditions are as follows: 56° C. for 30 min, 95° C. for 2 min, and 30 cycles of denaturing at 95° C. for 15 sec, annealing at 56° C. for 30 sec, and extension at 68° C. for 40 sec with a final extension at 68° C. for 7 min. FIG. 1 demonstrates detection of FCGR2C mRNA from individuals with different allelic display demonstrating subjects that express mRNA for full length FcγRIIc. Further, B-cells express no detectable FcγRIIa but plentiful FcγRIIb and FcγRIIc. In contrast, control U397 cells express abundant FcγRIIa and FcγRIIb, but no detectable FcγRIIc.

Alternatively, total cellular RNA is isolated from all cell types using the RNA-Bee (AMS Biotechnology, Ltd, United Kingdom) process. Cell types include B-cells, T-cells, NK cells, neutrophils, dendritic cells, and monocytes/macrophages that are cultured or enriched from subjects. Operable cell lines are illustratively available from American Type Culture Collection (ATCC) (Manassas, Va.). cDNAs of FcγRIIa, FcγRIIb, FcγRIIc, or other target genes are synthesized by reverse transcriptase (RT) from 2 μg of total RNA isolated from each cell source using a ProtoScript® First Strand cDNA Synthesis Kit (New England Biolabs, Ipswich, Mass.). The cDNA is amplified by PCR to detect the target genes of interest. Alternatively, TaqMan real-time RT-PCR is used to amplify the target mRNA of interest as per the manufacturers protocol. Probes for FcγRII isoforms are as described by Metes, 1998, incorporated herein by reference or synthesized to anneal to any desired sequence.

### Example 2

Detection of FcγRII proteins from various cell types. Processes of immunoprecipitation and western blotting are performed as illustratively described by Su, 2007. Target cells are cultured or obtained from subjects, optionally enriched and isolated as described in Example 1. Resulting cells are lysed with whole cell lysis buffer using a final cell concentration of 60 μl/1×10⁶ cells. Li, X, et al, Arthritis Rheum, 2003; 48:3242-3252, incorporated herein by reference. Lysis solutions are vortexed for 10 sec and incubated on ice for 30 min with a brief vortexing every 10 min. The samples are centrifuged at 15,000 rpm at 4° C. for 15 min and the supernatant collected. For immunoprecipitation, monoclonal antibodies (mAbs) 4F5, IV.3, or AT-10 are added to the whole cell lysate and incubated at 4° C. for 2 h with mixing. Protein G Sepharose beads are added to each sample and the samples further incubated at 4° C. for 1 h with mixing. Beads are washed 4 times with whole cell lysis buffer and the immunoprecipitates are subjected to western blot analysis for detection and quantification. Target FcγRII isotype protein is quantified by western blot based on a standard curve of known quantities of the desired FcγRII isotype.

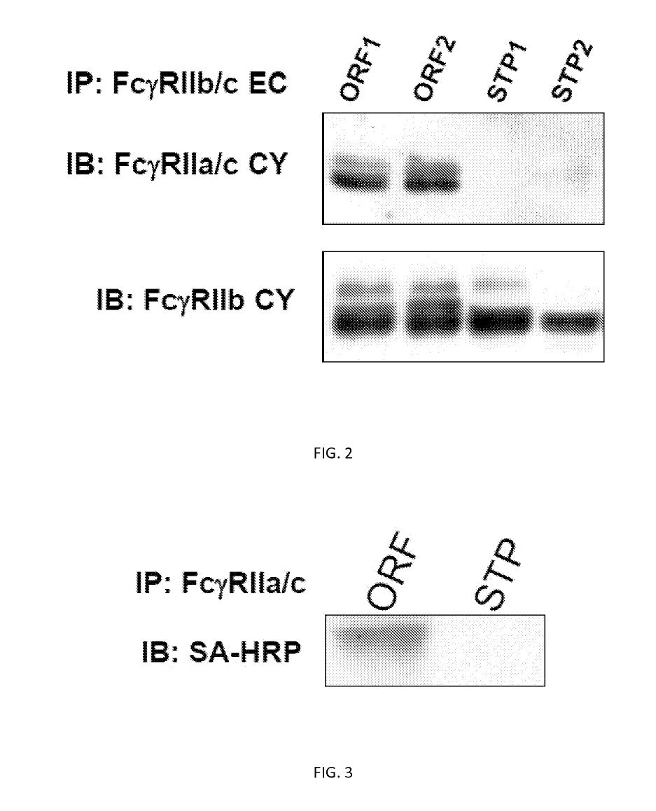

FIG. 2 illustrates immunoprecipitation from B-cells of both FcγRIIb and FcγRIIc and subsequent detection of either FcγRIIb or FcγRIIc. The FcγRIIb/c extracellular domain is immunoprecipitated with antibody 4F5 (Su, 2007). Alternatively, FcγRII isotypes are simultaneously immunoprecipitated by pan-mAb AT10 as described by Su, 2004. Western blot for FcγRIIa/c cytoplasmic tails is performed with anti-FcγRIIa/c blotting antibodies (Santa Cruz Biotechnology, Santa Cruz, Calif.). Id. Antibody E-16 (Santa Cruz Biotechnology, Santa Cruz, Calif.) is used for detection of FcγRIIb by western blot. FIG. 2 illustrates the presence of protein in B-cells from subjects homozygous for FcγRIIc open reading frame (ORF) allele whereas subjects with the pseudogene for FcγRIIc show no protein expression.

Alternatively, expression of FcγRII isoforms on a cell surface are detected by biotinylation of cell surface proteins, lysis of the cells, immunoprecipitation by FcγRIIc isotype specific antibodies and detection using avidin-HRP. FIG. 3 illustrates expression of FcγRIIc isotype on the surface of circulating B-cells from subjects expressing FcγRIIc ORF.

### Example 3

Detection of FcγRII isoforms on multiple cell types by flow cytometry. IV.3 and 32.2 hybridomas are purchased from American Type Culture Collection (ATCC, Manassas, Va.). The AT10 hybridoma is as described. Greenman, J. et al., Mol Immunol, 1991; 28:1243-1254. Antibody fragments are prepared by Rockland Immunochemicals, Inc., Gilbertsville, Pa. Antibody 4F5 is prepared as described by Su, 2004. Antibodies are conjugated with A1exa488, Cy-5, or FITC fluorescence dye using a corresponding Protein Labeling Kit (Invitrogen-Molecular Probes, Carlsbad, Calif.). The labeling efficiency of all antibodies is determined following the manufacturer's instructions, and is approximately 3:1 (3 molecules of dyes per protein molecule). Anti-FcγRIIa/c antibody conjugated with Cy5-phycoerethrin is used for detection of FcγRIIc. Antibody 4F5 conjugated to FITC is used for detection of FcγRIIb. An ImmunoPure Fab Preparation Kit (Pierce Biotechnology, Rockford, Ill.) is used to prepare control F(ab′)₂ fragments. As described in Su, 2004, CD19-APC and CD14-TRI-COLOR mAbs are available from Caltag Laboratories (Burlingame, Calif.). CD27-APC mAb are available from e-Biosciences (San Diego, Calif.). BDCA1-APC and BDCA2-APC mAbs are available from Miltenyi Biotec (Auburn, Calif.). The isotype control mIgG and mIgG F(ab′)₂ and F(ab′)₂ goat anti-mouse IgG F(ab′)₂ are available from Jackson ImmunoResearch Laboratories (West Grove, Pa.). Goat polyclonal antibodies specific for the cytoplasmic domain of FcγRIIa/c are available from Santa Cruz Biotec (Santa Cruz, Calif.).

Cells are optionally enriched as described in Example 1. Enrichment is not absolutely required, but may provide more robust signal and specific detection. PBMCs or whole blood are incubated with the indicated mAbs for 45 min on ice. Anti-FcγRIIa/c antibody conjugated with Cy5-phycoerethrin is used for detection of FcγRIIc. Antibody 4F5 conjugated to FITC is used for detection of FcγRIIb. The cells are washed with 3 ml of ice-cold PBS plus 0.5% BSA and 0.02% NaN₃. The red blood cells are lysed by incubation with 1.5 ml 1×FACS Lysing Solution (BD Biosciences, San Jose, Calif.) at room temperature for 15 min. The cells are washed with PBS and resuspended in PBS plus 1% paraformaldehyde for flow cytometry analysis. Flow cytometry analysis of stable transfectants is performed similarly without the red blood cell-lysing step.

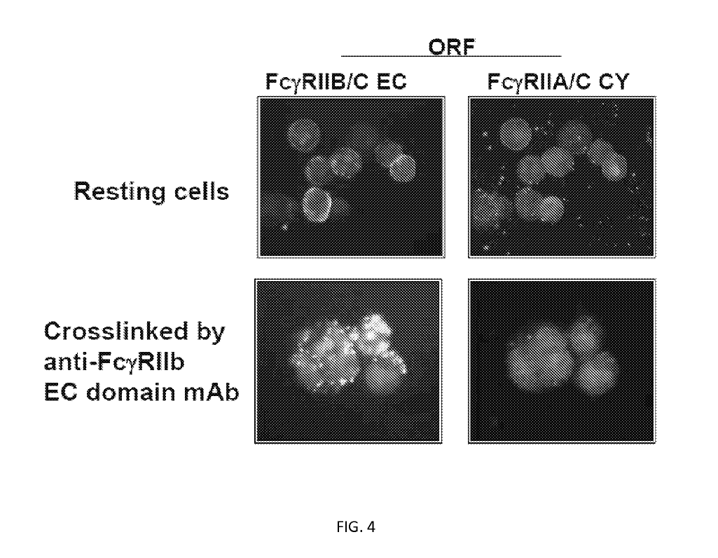

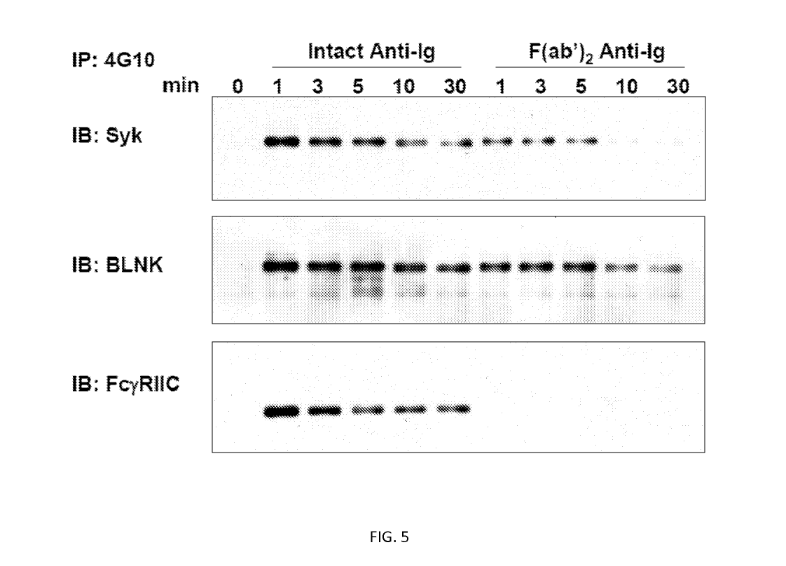

FIG. 4 illustrates expression of both FcγRIIb and FcγRIIc on B-cells enriched and prepared as in Example 1. The top panels depict surface expression of either both FcγRIIb and FcγRIIc isoforms or just FcγRIIc (right panels; B-cells do not express FcγRIIa). Cros slinking of the FcγRII receptors by a monoclonal anti-FcγRIIb antibody produces clustering of the receptors on the B-cell surface.

### Example 4

Quantification of FcγRII isoforms on multiple cell types. Processes for detecting and determining an immune response in numerous cell types is described in WO/2005/085864 which is incorporated herein by reference. Processes for quantifying the expression level of FcγRII isoforms on numerous cell types is accomplished substantially as described by Mauer, KJ, et al, Clin Diagn Lab Immunol, 2002; 9:1248-1252 incorporated herein by reference.

Alternatively, a radioimmunoassay is employed. Radioimmunological processes are substantially as described by Williams, TE, et al, Biophys J, 2000; 79:1858-1866 incorporated herein by reference. Antibodies directed to the extracellular domains of FcγRII isoforms are as described in the prior examples. Antibodies, or F(ab′)₂ fragments thereof, are labeled with ¹²⁵I using IODO-GEN Precoated Reaction Tubes (Pierce, Rockford, Ill.) as per the manufacturer's instructions with are incorporated herein by reference. ¹²⁵I-Fab of a selected antibody is incubated with cells at a saturating concentration. Samples are analyzed in triplicate. After all samples are thoroughly washed and counted, the cell-bound radioactivity is measured on a gamma counter. Readings are converted to site densities using the previously measured and known specific activity of the ¹²⁵I-Fab directed at the selected target FcγII receptor, and determined in parallel from standard samples (˜2 μl aliquots of ¹²⁵I-Fab). The mean expression level for FcγRIIb on B-cells is 9.3×10⁵ per cell.

### Example 5

Activation of cell signaling molecules in response to FcγRIIc activation. A2011A1.6 cells, which are devoid of Fc receptor expression, are transfected with the pcDNA3 vector (Invitrogen Corp., Carlsbad, Calif.) into which FcγRIIc (OCR) is subcloned. Analyses of signaling protein phosphorylation is performed as described by Haga C L, et al, Proc Nati Acad Sci USA. 2007; 104:9770-9775 incorporated herein by reference. Cells (5×10⁶) are washed twice with PBS and incubated for 2 h in FCS deficient media buffered with 20 mM Hepes (pH 7.2). BCR and FcγRIIc are co-ligated by stimulation with intact anti-IgG antibodies (25 μg/ml) or anti-IgG F(ab′)₂ fragments (16.6 μg/ml). Activated and control samples are lysed with M-PER cellular lysis buffer (Pierce Biotechnology, Rockford, Ill.) supplemented with Complete Protease Inhibitor (Roche Applied Sciences, Indianapolis, Ind.), and the phosphatase inhibitors Na₃VO₄ (0.2 mM), Na₂MoO₄ (1 mM), and β-glycero-phosphate (5 mM). Total protein concentration is quantified using the bicinchoninic acid solution (BCA) reagent (Pierce Biotechnology, Rockford, Ill.) as per the manufacturer's protocol, which is incorporated herein by reference. Whole cell lysates are incubated for 1 h at 4° C. with 20 μl of 50% slurry of protein A-sepharose beads (Invitrogen Corp., Carlsbad, Calif.) conjugated to the anti-phosphotyrosine antibody 4G10 Platinum (Millipore, Billerica, Mass.) in PBS with gentle rocking. Beads are pelleted by centrifugation, the supernatants removed, and the beads washed 5 times with 1 ml of M-PER buffer and boiled. Western blotting is performed by techniques known in the art by separation using SDS-PAGE followed by transfer to nitrocellulose or PVDF membranes (MSI, Westboro, Mass.). Proteins are recognized by antibodies directed to Syk (C-20), BLNK (2B11), Btk (7F12H4), or FcγRIIc (4F5), and the proteins were visualized by using the ECL reagent (Amersham Pharmacia Biosciences, Piscataway, N.J.). Antibodies C-20, 2B11, and 7F12H4 are available from Santa Cruz Biotechnology, Santa Cruz, Calif.).

FIG. 5 depicts phosphorylation of FcγRIIc within 1 min following cros slinking of FcγRIIc and BCR. The phosphorylation level reaches a maximum between one and three minutes and is thereafter reduced over time. Coincident with the increase in FcγRIIc phosphorylation, the intracellular signaling molecules Syk and BLNK each are phosphorylated with a corresponding decrease in the level of phosphorylation over time along with that of FcγRIIc. Thus, crosslinking of FcγRIIc and BCR results in an increase in the activation of Syk and BLNK and enhances the magnitude of BCR activation.

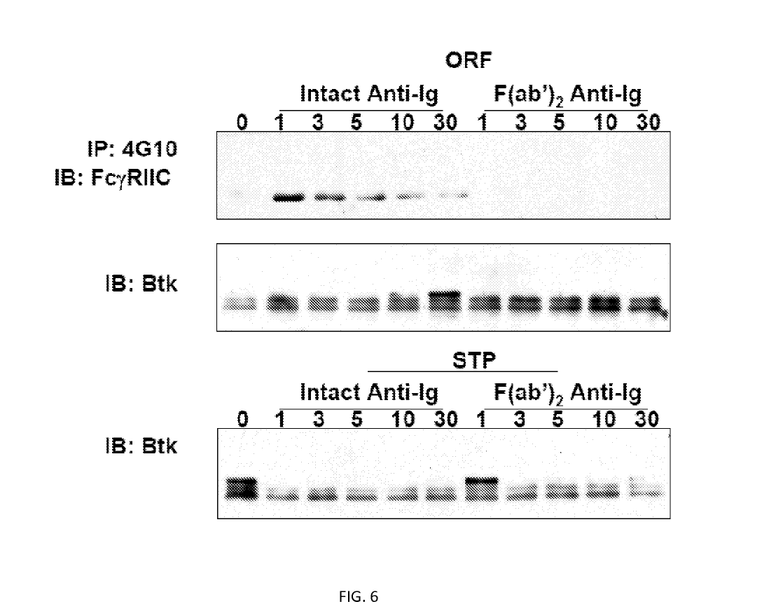

In B-cells expressing FcγRI1b, BCR induced activation of Btk is inhibited. FIG. 6 depicts a reduction in this inhibition following crosslinking of FcγRIIc and BCR. Thus, FcγRIIc reverses the inhibition of Btk activation.

### Example 6

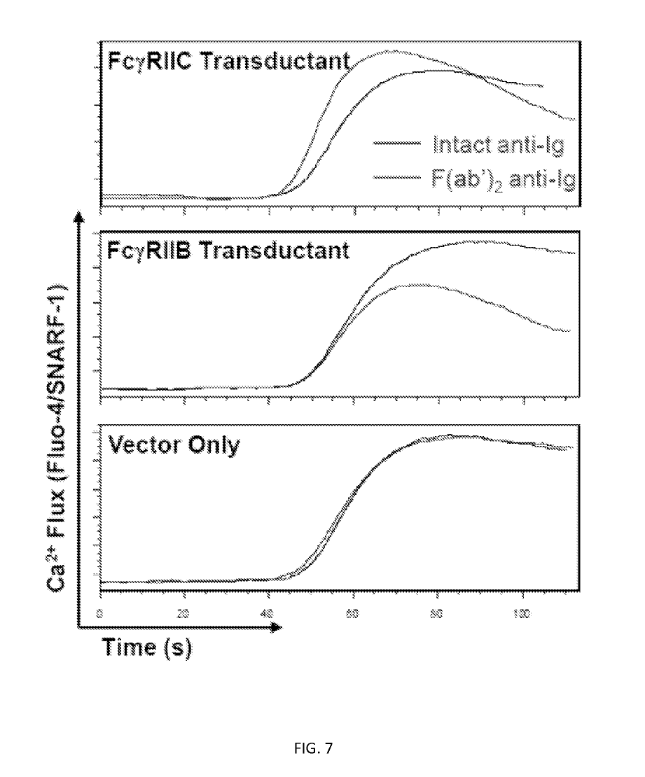

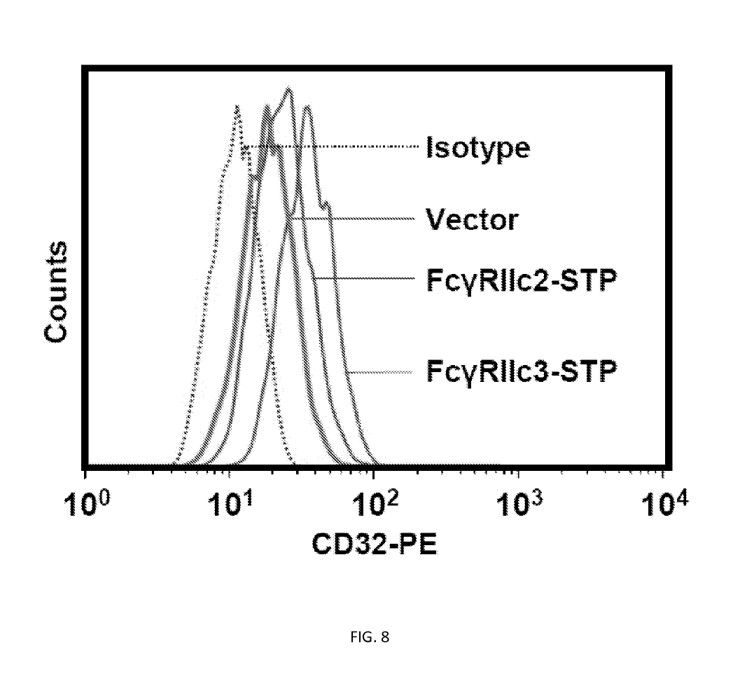

FcγRIIc reversed the inhibition of BCR induced Ca²⁺ flux associated with the presence of FcγRIIb. Ca²⁺ flux in cells is inhibited by crosslinking FcγRIIb and BCR. Ca²⁺ flux is measured as described by Haga, et al, Proc Nati Acad Sci USA. 2007; 104:9770-9775 incorporated herein by reference. B-cells (5×10⁶) isolated from subjects expressing either ORF FcγRIIc or the pseudogene are washed twice in Hanks' balanced salt solution (HBSS) (with Ca²⁺ and Mg²⁺), then resuspended in 500 μl of Fluo-4 NW assay buffer (Invitrogen Corp, Carlsbad, Calif.) and incubated at 37° C. for 30 min followed by 30 min at room temperature. Loaded cells (250 μl) are analyzed by fluorometry prior to and following addition of 25 μg/ml intact IgG or 16.6 μg/ml F(ab′)₂ fragments. Crosslinking of FcγRIIc and BCR reduces the inhibition of Ca²⁺ flux or mobilization in immune cells as depicted in FIG. 7.

### Example 7

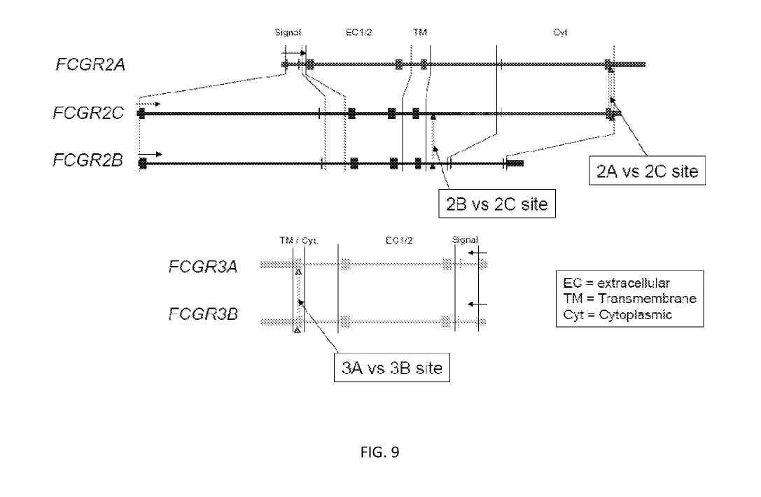

Determining a level ratio classification system and binary and ternary ratios for FcRγIIa, FcRγIIb, and FcRγIIc. The relative abundance of copy number for genes encoding FcRγIIa, FcRγIIb, and FcRγIIc is achieved by pyrosequencing methodologies. The binary ratios of FcRγIIa:FcRγIIb, FcRγIIb:FcRγIIc as well as the ternary ratio of FcRγIIa:FcRγIIb:FcRγIIc is achieved by capitalizing on single nucleotide intergenic sequences present in each of the genes. As illustrated in FIG. 9, a characteristic FcRγIIa to FcRγIIc site is present that allows sequence differentiation. Similarly, a characteristic FcRγIIb to FcRγIIc site is present that allows sequence differentiation. Pyrosequencing of the amplified regions of interest reveals relative copy numbers of each gene in the subject.

The pyrosequencing method is performed in a 96-well plate format in the PSQ HS96 instrument (Qiagen) that can run up to 10 plates at a time. Following PCR amplification of the targeted region, one strand of the amplicon is purified through the use of a biotinylated PCR primer and a sequencing primer is then hybridized to the isolated strand. Nucleotides are added sequentially to each well and the amount of incorporation is quantitated to determine the genotype in each well. Controls include the use of a no template control well in every plate.

Using a nested PCR strategy, an initial Fcγ receptor gene specific reaction is performed that ranges in size from 1.5 kB to over 15 kb depending on the location of the variant within the Fcγ receptor gene. First round PCR reactions contain 25-50 ng of template DNA, 1.5U Taq polymerase, 0.01 μM of each primer, 0.2 mM dNTP, 1.5 mM of MgCl₂ and 20 mM of Tris-HCl (pH 8.4) and 50 mM KCl in a 25 μl volume. Second round nested PCR reactions around the SNP of interest are then performed on the first round gene specific amplicons. Similar PCR conditions are used except for the use of 0.25-0.5ul of first round PCR product in place of genomic DNA and the use of a biotinylated PCR primer to allow for strand purification for sequencing. All PCR reactions are run in ABI9700 PCR machines (Applied Biosystems).

Human FcRγIIa and FcRγIIb spanning the nucleotide intergenic sequences site is amplified by polymerase chain reaction (PCR) from 423 known normal patients and 308 SLE diagnosed patients. The reverse primer is 5′-biotinylated to facilitate single-strand DNA template isolation for the pyrosequencing reaction. Primers are synthesized by Integrated DNA Technologies (Coralville, Iowa). Each PCR reaction contains 20 to 50 ng of genomic DNA, 10 μmol of each primer, and 25 μl of Jumpstart Readymix REDTaq polymerase (Sigma, St. Louis, Mo.) in a total volume of 50 μl. Cycling is performed in an Eppendorf Mastercycler Gradient (Brinkman Instruments, Westbury, N.Y.). For sequencing of the FGCR2C gene, a first round gene-specific Long PCR reaction is carried out to specifically amplify a 6277 bp region encompassing the SNP 202T/C (sense primers: 5′-CTGCATATGTTGTCCCCCTGTGTTGCTAAAT-3′ (SEQ ID NO: 6); antisense primer: 5′-AACATGAGAGAGAAAAAGAGAGGCAGGGAGGGAGCTTA-3′ (SEQ ID NO: 7). Using High Fedelity PCR kit, the conditions are: 94° C. for 2 min, and 35 cycles of denaturing at 94° C. for 30 s, annealing at 60° C. for 30 s, and extension at 68° C. for 7 min 30 s with a final extension at 68° C. for 7 min); the 6 kb PCR product is then used as template for a nested PCR reaction to amplify a short fragment of the FCGR2C gene containing the 202T/C site (sense primer: 5′-GGCCTACAGGTGCTTTTTTGTCT-3′ (SEQ ID NO: 8), antisense primer: 5′-biotin-AGTCGCTCTCAGGGCTGTAAGT-3′ (SEQ ID NO: 9), PCR conditions are as follows: 94° C. for 3 min, and 40 cycles of denaturing at 94° C. for 30 s, annealing at 56° C. for 30 s, and extension at 72° C. for 20 s with a final extension at 72° C. for 7 min); finally this fragment is pyrosequenced to determine the genotype of FCGR2C (primer: 5′-TGTGCTGAAACTCGAGCC-3′) (SEQ ID NO: 10).

Within the FCGR2A and FCGR2C genes, the single base difference (noted as [C/T]) between the genes together with flanking sequence, is: AACGTTATGCCATGTGGTCA [C/T] ACTCTCAGCTTGCTGAGTGG (SEQ ID NO: 11).

Within the FCGR2B and FCGR2C genes, the single base difference (noted as [T/C]) between the genes together with flanking sequence, is: GTGGAAAATGGGGACACTAA [T/C] AGGACTTACCTCAGAGGGTT (SEQ ID NO: 12).

Successful and specific amplification of the region of interest is verified by visualizing 5 μl of the PCR product on a 2% agarose gel containing ethidium bromide.

Following PCR, single stranded PCR products are purified from 8-10 μl of second round PCR reaction using the biotinylated primer and immobilization to streptavidin beads with the PyroMark Vacuum Prep Workstation (Qiagen, Valencia, Calif.), denatured with NaOH and annealed to the sequencing primer by heating to 80° C. for 2 min. Pyrosequencing reactions are performed according to the manufacturer's instructions on a PSQ-HS96A system (Qiagen). The template is incubated with 0.4 μmol/L sequencing primer at 80° C. for 2 minutes

As nucleotides are dispensed, a light signal is generated proportional to the amount of each incorporate^{d} nucleotide. These light signals are detected by a charge-couple^{d} device camera and converted to peaks in a sequencing pyrogram that is automatically generated in real time for each sample Dideoxy sequencing of each sample is optionally performed for increased confidence.

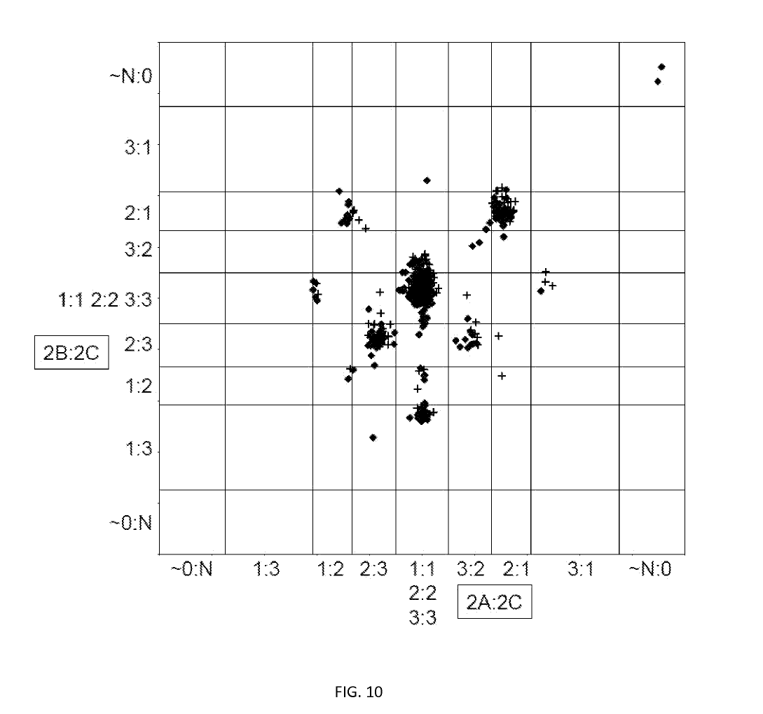

The results of the relative copy numbers are plotted in a two dimensional array. As depicted in FIG. 9, binary or ternary protein ratios are established for control patients. FIG. 10 also depicts the ratio pattern observed for SLE patients. By comparison of an unknown sample from a patient to the established binary or ternary ratios of the FcγRII receptors an investigator is able to predict the propensity of an immune response in the unknown subject. Illustratively, the presence of increased FcγRIIc levels relative to FcγRIIb reveals in inhibited propensity for BCR signaling or reduced inhibition of Ca²⁺ flux or mobilization in immune cells. Similarly, a baseline therapeutic response is established in control patients and patients with various copy level ratios and ratio patterns to predict the magnitude, extent, duration, or lag time among others from administration of a therapeutic.

### Example 8

Construction of protein classification system, binary ratio, and ternary ratio. A Luminex assay platform (Luminex, Corp., Austin, Tex.) is used to create a protein classification system for the three Fc receptors FcγRIIa, FcγRIIb, and FcγRIIc. Beads with fluorescence F1 and F2 are individually coated either with capture anti-FcγRIIa/c antibody directed to the intracellular domain of FcγRIIa/c or antibody 4F5 directed to the extracellular domain of FcγRIIb/c respectively as per the manufacturer's protocol.

For determination of the FcγRIIb/c protein ratio, the F2 bead is used as a capture support. Detection of FcγRIIb is achieved by Cy5 conjugated antibody C-20 (Santa Cruz Biotechnology, Santa Cruz, Calif.) directed to the intracellular domain of FcγRIIb. Detection of FcγRIIc is achieved by Oregon Green conjugated anti-FcγRIIa/c antibody directed to the intracellular domain of FcγRIIa/c. All antibodies are used at saturating concentrations. Antibodies are conjugated to fluorophores using a corresponding Protein Labeling Kit (Invitrogen-Molecular Probes, Carlsbad, Calif.). Fluorescence intensities are normalized based on a fluorescence standard curve.

The value classification systems are obtained by screening 100 normal control patients. B-cells are isolated as described in Example 1. The resulting cells are counted and lysed as described in Example 2 to solubilize the transmembrane proteins. A protein sample is incubated with either F1 or F2 beads coated with the respective capture antibody for 2 hours with gentle rocking. The beads are washed and blocked with BSA. Beads are washed and detection antibodies C-20 and anti-FcγRIIa/c antibody are incubated with the sample for 1 hour in the dark with gentle rocking. After a final wash the relative abundance of FcγRIIb/c is determined by flow cytometry. A distinguishable pattern of FcγRIIb/c is obtained for normal and patients diagnosed with either RA or SLE. Thus, an unknown subject matching or similar to one pattern can be diagnosed for the presence or absence of disease. Similar profiles are created for a set of individuals prior to and following dosing with a therapeutic. The known response to the therapeutic is used to plot the protein ratio classification system. Also, a known immune response for subjects is used to generate a protein ratio classification system based on the presence or absence of the immune response or magnitude thereof. Distinguishable patterns are obtained for each classification system.

The binary ratio of FcγRIIb/c for an unknown subject is achieved using the identical protocols. Comparison of the resulting binary protein ratio is compared with the value classification system to predict an immune response, a response to a therapeutic, or diagnosis of a disease.

Similar protocols are used to generate a ternary value classification system and ratios. The relative level of FcγRIIa/c is determined using F1 beads coated with capture antibody anti-FcγRIIa/c. The differential detection antibodies are anti-FcγRIIa EC domain ZZ18 (Santa Cruz Biotechnology, Santa Cruz, Calif.) conjugated to Cy5 and anti FcγRIIc EC domain antibody 4F5 conjugated to Oregon Green. The relative levels of FcγRIIa/c are compare to FcγRIIb/c to generate the ternary ratio FcγRIIa/b/c. The value classification systems for disease diagnosis for RA and SLE are obtained from 100 normal and 100 diagosed patients with RA or SLE. Classifications systems are generated for response to therapeutics and immune responses as well. The ternary ratio from an unknown subject are compared to the value classification of interest and the presence or absence of RA or SLE is diagnosed, the predicted response to a therapeutic is generated or the predicted immune response is predicted by identifying matching or nearly matching profiles.

### Example 9

mRNA classification system and ratio determinations. Human B cells are prepared as in Example 1. Cells are lysed and total cellular RNA is isolated using the RNAzo1 B (Biotex Labs Inc, Houston, Tex.) method. cDNAsa synthesized by RT from 2 μg of total RNA isolated from each cell source using a first-strand cDNA synthesis kit (Pharmacia-Biotech).

Forward and reverse primers flanking the site of unequal crossover defining FcγRIIa, FcγRIIb, and FcγRIIc are used to amplify each mRNA. Species specific probes that traverse the crossover site are used to differentially detect the amplification of each mRNA species. The absolute and relative levels of mRNA for each FcγRII species is detected by real-time PCR.

Samples from 100 subjects of known diagnosis are screened for relative mRNA levels to generate mRNA ratio value classification systems for disease diagnosis, immune response, or response to therapeutic. The mRNA profiles for expression of FcγRIIb, FcγRIIc, and/or FcγRIIa are obtained for a unknown and compared to the previously determined value classification system corresponding to the desired output.

Various modifications of the present invention, in addition to those shown and described herein, will be apparent to those skilled in the art of the above description. Such modifications are also intended to fall within the scope of the appended claims.

Patents and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

The following references are each incorporated herein by reference as if the contents of each reference were fully and explicitly included.

### REFERENCE LIST

1. Dijstelbloem H M, et al. Trends Immunol, 2001; 22:510-516
2. Salmon, J E, and Pricop, L, Arthritis Rheum, 2001; 44:739-750
3. Ravetch, J V, and Bolland, S, Annu Rev Immunol, 2001; 19:275-90
4. Wijngaarden, S, et al, Arthritis Rheum, 2004; 50:3878-3887
5. Mellman, I, and Steinman, R M, Cell, 2001; 106:255-258
6. Mulherin, D. et al, Arthritis Rheum, 1996; 39:115-124
7. Burmester, G R, et al, Arthritis Rheum, 1997; 40:5-18
8. Tak P P, et al, Arthritis Rheum, 1997; 40:215-225
9. Winchester, R J, et al, Clin Exp Immunol, 1970; 6:689-706
10. Mannik, M, J Rheumatol Suppl, 1992; 32:46-49
11. Hulett, M D, and Hogarth, P M, Adv Immunol, 1994; 57:1-127
12. Daeron, M, et al, J Immunol, 1992; 149:1365-1373
13. Daeron, M, et al, Immunity, 1995; 3:635-646
14. Brooks, D G, et al, J Exp Med, 1989; 170:1369-1385
15. Qiu W Q, et al, Science, 1990; 248:732-735
16. Warmerdan P A M, et al, J Biol Chem, 1993; 268:7346-7349
17. Cassel, D L, et al, Mol Immunol, 1993; 30:451-460
18. Ravetech, J V, and Kinet J P, Annu Rev Immunol, 1991; 9:457-492
19. Capel, P J A, and van de Winkel, J G J, Immunol Today, 1993; 14:215-221
20. Hulett, M D, and Hogarth, M, Adv Immunol, 1994; 57:1-127
21. Ravetch, J V, Cell, 1994; 78:533-560
22. Lin, C T, et al, J Clin Immunol, 1994; 14:1-13
23. Metes, D, et al, Blood, 1998; 91:2369-2380
24. Odin, J A, et al, Science, 1991; 254:1785-1788
25. Tuijnman, W, et al, Blood, 1992; 79:1651-1656
26. van den Herik-Oudijk, I E, et al, J Immunol, 1994; 152:574-585
27. Muta, T, et al, Nature, 1994; 368:70-73 (Erratum in: Nature, 1994; 369:340)
28. Su, K, et al, J Immunol, 2004; 172:7186-7191
29. Dhodapkar, K M, et al, J Exp Med, 2007; 204: 1359-1369
30. Stavenhagen, J B, et al, Cancer Res, 2007: 67:8882-8890
31. Omori, S A, and Wall, R, PNAS USA, 1993; 90:11723-11727
32. Rao, M K, and Wilkinson, M F, Nature Protocols, 2006; 1:1494-1501
33. Kenrick, M K, et al, Nucleic Acids Res, 1995; 25: 2947-2948
34. Stuart, S G, et al, J Exp Med, 1987; 166:1668-1684
35. Stuart, S G, et al, EMBO J, 1989; 8:3657-3666
36. Warmerdam, P A M, et al, J Exp Med, 1990; 172:19-25
37. Su, K, et al, J Immunol, 2007; 178:3272-80
38. Li, X, et al, Arthritis Rheum, 2003; 48:3242-3252
39. Greenman, J. et al., Mol Immunol, 1991; 28:1243-1254
40. Mauer, K J, et al, Clin Diagn Lab Immunol, 2002; 9:1248-1252
41. Williams, T E, et al, Biophys J, 2000; 79:1858-1866
42. Haga C L, et al, Proc Nall Acad Sci USA. 2007; 104:9770-9775
43. Strome, S E, et al, The Oncologist, 2007; 12:1084-1095
44. Short Protocols in Molecular Biology, 5th Edition, John Wiley & Sons, Inc., Hoboken, N.J.

1. A process of altering an immune response in a subject comprising:
regulating FcγRIIc protein expression or FcγRIIc protein activity.

2. The process of claim 1 wherein said regulating is altering said FcγRIIc protein expression.

3. The process of claim 2 wherein said regulating is increasing said FcγRIIc protein expression.

4. The process of claim 2 wherein said regulating is decreasing said FcγRIIc protein expression.

5. The process of claim 1 wherein said regulating is altering said FcγRIIc protein activity.

6. The process of claim 5 wherein said altering is increasing said FcγRIIc protein activity.

7. The process of claim 5 wherein said altering is decreasing said FcγRIIc protein activity.

8. The process of claim 1 wherein said FcγRIIc protein is expressed on a surface of a B-cell, T-cell, dendritic cell, monocyte, macrophage, or natural killer cell.

9. The process of claim 1 wherein said FcγRIIc protein is expressed on a B-cell.

10. A process of predicting an immune response in a subject comprising:
determining the level of FcγRIIc in a cell of the subject;
determining the level of FcγRIIb in said cell;
calculating a binary immune ratio of said FcγRIIb/FcγRIIc for said cell;
determining a FcγRIIb/FcγRIIc ratio value classification system from a plurality of subjects of known immune response; and
predicting an immune response from comparing said binary immune ratio and said FcγRIIb/FcγRIIc ratio value classification system.

11. The process of claim 10 further comprising:
determining the level of FcγRIIa in said cell;
calculating a ternary immune ratio of said FcγRIIa/FcγRIIb/FcγRIIc for said cell; and
determining a FcγRIIa/FcγRIIb/FcγRIIc ratio value classification system from a plurality of subjects of known immune response; and
predicting an immune response from comparing said ternary immune ratio and one or more values of said FcγRIIa/FcγRIIb/FcγRIIc ratio value classification system.

12. The process of claim 10 or 11 wherein said cell is one of a B-cell, T-cell, a dendritic cell, a monocyte, or a macrophage.

13. The process of claim 12 wherein said cell is immortalized.

14. A process of predicting response to a therapeutic in a subject comprising:
determining the level of FcγRIIc in a cell of the subject;
determining the level of FcγRIIb in said cell;
calculating a binary ratio of said FcγRIIb/FcγRIIc for said cell;
determining a FcγRIIb/FcγRIIc ratio value classification system from a plurality of subjects of known response to the therapeutic; and
predicting the response to the therapeutic by comparing said binary immune ratio and one or more values of said FcγRIIb/FcγRIIc ratio value classification system.

15. The process of claim 14 further comprising:
determining the level of FcγRIIa on said cell;
calculating a ternary ratio of FcγRIIa/FcγRIIb/FcγRIIc protein for said cell; and
determining a FcγRIIa/FcγRIIb/FcγRIIc ratio classification system from a plurality of subjects of known response to the therapeutic; and
predicting the response to the therapeutic from comparing said the ternary ratio and one or more values of said FcγRIIa/FcγRIIb/FcγRIIc ratio value classification system.

16. The process of claim 14 or 15 wherein said therapeutic is selected from the group comprising: rituximab, monoclonal antibody, polyclonal antibody, antibody fragment, GM-CSF, other cytokines, interleukins, or combinations thereof.

17. A process of diagnosing disease or condition in a subject comprising:
obtaining a biological sample from the subject,
isolating a cell from the subject;
determining the level of FcγRIIb and level of FcγRIIc in said cell from the subject;
calculating a binary ratio of FcγRIIb/FcγRIIc for the cell;
determining a FcRγIIb/FcRγIIc ratio value classification system; and
diagnosing the disease or the condition in the subject based on comparing the binary ratio and one or more values of the FcRγIIb/FcRγIIc ratio value classification system.

18. The process of claim 1 or 10 or 14 further comprising determining a FcγRIIb haplotype for the subject.

19. The process of claim 18 wherein the level FcγRIIc is determined in a B-cell.
